(19) United States
(12) Patent Application Publication
(10) Pub. No.: US 2024/0353430 A1
(43) Pub. Date: Oct. 24, 2024

(54) OPACITY TESTING THROUGH MEASURED ILLUMINANCE

(71) Applicant: Worcester Polytechnic Institute, Worcester, MA (US)

(72) Inventors: Kathryn Woodland, Boxford, MA (US); Madison Gass, Barrington, NH (US); Isabel Nearing, Cochecton, NY (US); Grace Jolin, Manchester, NH (US); Ziya Isiksacan, Boston, MA (US); Mathias Bonmarin, Worcester, MA (US); Sakthikumar Ambady, Marlborough, MA (US); O. Berk Usta, Andover, MA (US); Ahmet C. Sabuncu, Shrewsbury, MA (US)

(21) Appl. No.: 18/640,521

(22) Filed: Apr. 19, 2024

Related U.S. Application Data
(60) Provisional application No. 63/460,827, filed on Apr. 20, 2023.

(51) Int. Cl. G01N 33/72; G01N 21/78

(52) U.S. Cl. CPC G01N 33/726; G01N 21/78

(57) ABSTRACT

A storage and monitoring device for red blood samples in storage for donation (blood bank) and research. Stored blood can be susceptible to degradation, most often from a phenomenon known as ice nucleation. Accurate detection and identification of relevant factors causing ice nucleation can determine healthy samples of stored blood, and identify storage factors that promote storage longevity. A photodetection approach directs an illumination source and photometer on opposed sides of a blood containing vessel for measuring an illuminance affected by hemoglobin released from ruptured cells. Hemoglobin released from ruptured cells reduces light passage though the sample to indicate unusable samples. An accelerometer and temperature sensor measure the physical and temperate factors correlating with the detected cell degradation to determine storage criteria for extending blood longevity.

### RELATED APPLICATIONS

This patent application claims the benefit under 35 U.S.C. § 119 (e) of U.S. Provisional Patent App. No. 63/460,827, filed Apr. 20, 2023, entitled “OPACITY TESTING THROUGH MEASURED ILLUMINANCE,” incorporated herein by reference in entirety.

### STATEMENT OF FEDERALLY SPONSORED RESEARCH AND DEVELOPMENT

This invention was made with government support under grant No. 5R01HL145031, awarded by the National Heart Lung & Blood Institute, and grant No. 1941543, awarded by the National Science Foundation. The Government has certain rights in the invention.

### BACKGROUND

Red blood cells (RBCs) are one of the four components of whole blood, the other three being white blood cells, platelets, and plasma. They are responsible for transporting oxygen throughout the body, and bringing carbon dioxide to the lungs to be exhaled. RBCs are produced in the bone marrow and are round and flat, with an indent in the center. Hemoglobin is a protein within the cells that is responsible for the transport of oxygen. When RBCs undergo stress and are damaged, hemolysis occurs. As a result, the hemoglobin inside the cells leaks out due to rupturing of the cell.

### SUMMARY

A storage and monitoring device for red blood samples in storage for donation (blood bank) and research detects degradation of blood samples. Stored blood can be susceptible to degradation, most often from a phenomenon known as ice nucleation. Accurate detection and identification of relevant factors causing ice nucleation can determine healthy samples of stored blood, and can identify storage factors that promote storage longevity. A photodetection approach directs an illumination source and photometer on opposed sides of a blood containing vessel for measuring an illuminance affected by hemoglobin released from ruptured cells. Hemoglobin released from ruptured cells reduces light passage though the sample to indicate unusable samples. An accelerometer and temperature sensor measure the physical and temperate factors correlating with the detected cell degradation to determine storage criteria for extending blood longevity.

Configurations herein are based, in part, on the observation that blood storage, in conjunction with donor networks and distribution, is a vital process to medical, surgical and trauma treatment. Unfortunately, conventional approaches to blood banking and storage suffer from the shortcomings of cell longevity during prolonged storage. Particularly with rare blood types, an ability to store and transport blood to needy recipients is crucial. Accordingly, configurations herein substantially overcome the shortcomings of blood storage longevity by identifying hemoglobin degradation from ice nucleation in stored blood, and recording relevant factors affecting viable blood storage to avoid conditions unfavorable to blood longevity.

Ice nucleation causes supercooled red blood cell samples to deteriorate in quality, which is detrimental to researchers, doctors, and potential patients. Methods available for measuring cell lysis destroy the sample, leaving it unable to be used. A system that could detect when ice nucleation causes cell lysis in real time is needed. This technology can also be applied to storing samples other than red blood cells, ranging from tissue cells, proteins, or any deteriorating chemicals.

In further detail, the opacity testing device for a liquid blood sample includes a transparent vessel adapted for storing a liquid having an opacity, and an illumination source directed at a side of the vessel. A photometer is disposed on an opposed side of the vessel from the illumination source, and a processor connected to the photometer invokes nucleation logic for receiving an illuminance from the photometer, such that the illuminance is indicative of cell lysis. The processor computes a measure of healthy cells in the vessel based on the illuminance.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a blood sample suitable for use with configurations herein;

FIG. 2 is a schematic diagram of the device for blood sample evaluation and gathering of longevity data; and

FIGS. 3A and 3B show a comparison of healthy vs degraded blood samples.

### DETAILED DESCRIPTION

Configurations herein depict a device for evaluation of stored blood over time to detect a current state and an evolution of factors leading to the present state, whether healthy or unhealthy. Most stored blood undergoes some degradation even under optimal conditions, and is tolerable to some extent. The approach disclosed below depicts a method employed by the disclosed device for identifying when a blood sample has become degraded to a point of non-use, meaning it is unsuitable for use in patient transfusions.

It is commonly known and understood that blood is a vital component of human physiology. Consideration of blood as a treatment resource illustrate the cellular makeup. Red blood cells (RBCs) are one of the four components of whole blood, the other three being white blood cells, platelets, and plasma. They are responsible for transporting oxygen throughout the body, and bringing carbon dioxide to the lungs to be exhaled. RBCs are produced in the bone marrow and are round and flat, with an indent in the center. Hemoglobin is a protein within the cells that is responsible for the transport of oxygen. When RBCs undergo stress and are damaged, hemolysis occurs. As a result, the hemoglobin inside the cells leaks out due to rupturing of the cell.

Cryopreservation is the leading method of cell preservation due to its ability to store cells for long periods of time. The need for RBC transfusions is high, and access to a multitude of RBC units is necessary. Currently, cryopreservation is the only method of storage that allows for a large number of units, but transfusions cannot be performed immediately following preservation because the glycerol removal procedure is time consuming. If the glycerol cryoprotectant is not removed from the unit, intravascular hemolysis (lysis) can occur. Lysis refers to the breakdown of a cell caused by damage to its plasma (outer) membrane. It can be caused by chemical or physical means. Some researchers have begun to remove glycerol to determine the life span of the RBCs during deep supercooling in the hopes of preventing waiting times for transfusions. However, during deep supercooling, ice nucleation is a major cause of cell lysis in red blood cells.

Ice nucleation is characterized by the growth of ice crystals that cause irreparable damage to the cell and occurs during the freezing or thawing of the cells. The result is nucleation formation which causes the cells to lyse, and the samples become unviable. Reasons for this may include mechanical vibrations or temperature fluctuations, which indicates the need for a detection system to monitor the cells in real-time. Current ice nucleation detection methods often result in samples that are no longer viable and cannot be used for either transfusions or future research. Cell lysis measurement systems face this same issue. It would be beneficial to devise a system aimed at non invasive real time monitoring to ensure usable blood stock.

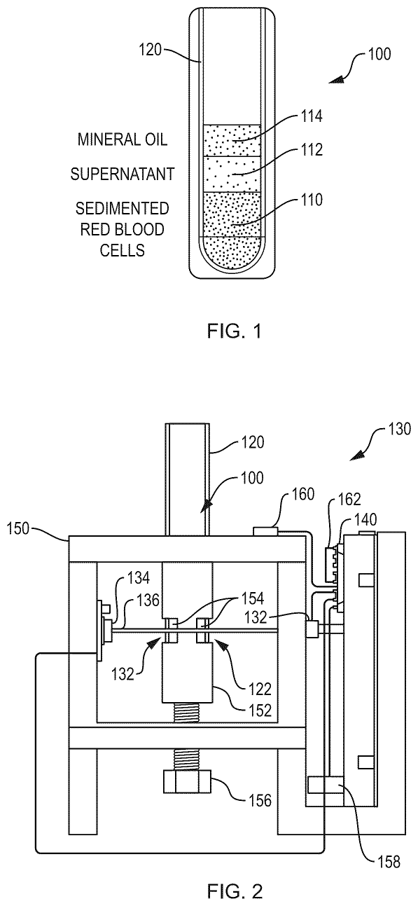

FIG. 1 is a context diagram of a blood sample 100 suitable for use with configurations herein, Referring to FIG. 1, the disclosed device monitors supercooled red blood cell samples to detect occurrences of ice nucleation. This device minimizes sample loss over conventional invasive testing methods for nucleation. A typical sample includes a sedimented red blood cell layer 110, a supernatant layer 112, and a layer 114 of mineral oil, stored in a vessel 120 such as a test tube. The supernatant layer 112 denotes the liquid lying above a solid residue after crystallization, precipitation, centrifugation, or other process—in this case above the sedimented red blood cell later 110.

When nucleation occurs, blood cells lyse and release hemoglobin; this causes the supernatant layer of the sample to change from a clear to a red color, which can be detected using a photometer. This photometer runs continuously with a vibration sensor and temperature sensor in order to monitor storage conditions, discussed further below.

Hemoglobin is widely considered to be the most important component of blood because of its ability to transport oxygen to the tissues. Hemoglobin is the protein that gives blood its characteristic red color. Structurally, hemoglobin contains two alpha protein chains and two beta protein chains. Each of these chains has a heme group, or a ringlike organic compound known as a porphyrin, attached to an iron atom, which allows for the vital transport of oxygen. The binding of oxygen to a hemoglobin molecule is a multi-step process. Once an initial oxygen molecule binds to one of the four heme groups in the molecule, the hemoglobin undergoes conformational change, which in turn enhances oxygen binding to the other three hemoglobin chains.

Supercooling, or long-term preservation at temperatures between −20° C. and 0° C., has shown promise in improvement of the blood storage standard, almost doubling the storage period. The main difficulty when supercooling samples is the formation of ice crystals within the solution, which in turn damages the red blood cells during supercooling. Deep supercooling provides an enhancement which seals the sample with an oil layer in order to prevent immediate ice formation. This is possible because mineral oil is molecularly composed of lipids, which vary in size and shape and prevent ice crystal formation. This extra layer of insulation surrounding the red blood cells and supernatant layer is expected to prevent freezing, but does not completely prevent spontaneous ice crystal formation, or ice nucleation. Ice nucleation starts at a nucleation site, such as an impurity or mechanical disturbance, that the ice starts to form around. Ice nucleation is another name for heterogeneous ice formation specifically when a sample is cooled below its freezing point. Nucleation refers to the nuclei or the initial location of the ice formation which can be another molecule or impurity or spontaneous formation of an organized pattern of the liquid molecules.

Configurations herein target the conditions under which ice nucleation degrades stored blood to the point of unusability. Liquids, like water, have molecules which are mobile and able to vibrate and move. Frozen liquids, or ice, have organized molecules which align and form a rigid lattice. The change from liquid to solid can happen in two major ways. The first being homogeneous, the formation of ice without a nucleation site. Homogeneous ice formation very rarely happens and never in biologic samples because water with no impurities is required. Then there is heterogeneous ice formation where there is a nucleation site, such as an impurity or mechanical disturbance, that the ice starts to form around. Ice nucleation is another name for heterogeneous ice formation specifically when a sample is cooled below its freezing point. Nucleation refers to the nuclei or the initial location of the ice formation which can be another molecule or impurity or spontaneous formation of an organized pattern of the liquid molecules.

The process of ice nuclei forming in a liquid is energetically favorable because the solid phase has higher free energy than the liquid. This is because the system is becoming more ordered and reduces the amount of entropy. The colder the liquid is, the less likely ice nucleation becomes due to the activity of the liquid molecules decreasing and therefore decreasing the odds of a stable ice nucleus forming.

FIG. 2 is a schematic diagram of the device for blood sample evaluation and gathering of longevity data. Referring to FIGS. 1 and 2, the disclosed approach employs an opacity testing device 130 for the liquid sample 100, including the transparent vessel 120 adapted for storing a liquid having an opacity, and an illumination source 132 directed at a side 122 of the vessel 120. A photometer 134 is disposed on an opposed side 132 of the vessel 120 from the illumination source 132. A processor 140 connects to the photometer 134 and an LED defining the illumination source 132. The processor is encoded with nucleation logic for receiving an illuminance from the photometer 134, where the illuminance is indicative of cell lysis, and computes a measure of healthy cells in the vessel based on the illuminance.

The vessel 120 is adapted to contain the sample 100 as in FIG. 1, where the sample having a plurality of layers of liquid. The illumination source 132 and the photometer 134 are disposed for aligning the illumination source and the photometer with a sensed layer of the plurality of layers, in this case the supernatant layer 112.

The device 130 has a form factor including a housing 150 and a receptacle 152 for containing the vessel 120. Typically the vessel has an elongated cylindrical shape and the receptacle 152 has a corresponding cylindrical void. Gaps 154 or voids in the receptacle 152 permit passage of a light beam 136 from the illumination source 132 through the transparent vessel 120 to the photodetector 134. As indicated above, the vessel 120 is adapted to contain a sample, such that an opacity of the supernatant layer 112 is based on a hemoglobin presence in the sample 100.

As volumes of the sample may vary, the housing employs a height control 156, such that the vessel 120 is responsive to the height control 156 for aligning the illumination source 132 and the photometer 134 with the supernatant layer 112 and the gaps 154 to permit unobstructed passage of the light beam 136 through the hemoglobin-tinted supernatant layer 112. The gaps 154 are of a height to likewise accommodate variances of the position of the supernatant later 112. A threaded engagement or similar mechanism of the height control 156 raises and lowers the receptacle 152.

The use of the light beam 156 provides an important feature of detecting ice nucleation without actually inciting it during storage, or otherwise damaging the sample during evaluation. The housing 150 also employs an accelerometer 158 and a temperature sensor 160 connected to the processor 140. A memory 162 is responsive to the accelerometer 158 and the temperature sensor 160 for detecting physical aberrations and a temperature corresponding to the illuminance at a particular time. Tracking of the sample 100 and parameters of illuminance, temperature and agitation or vibration provides a history of conditions under which nucleation tends to be facilitated or prevented. The memory 162 is coupled to the processor 140, and includes a table of values. The table of values is for receiving a value of illuminance from the photometer; and mapping the value of illuminance to a hemoglobin concentration, such that the hemoglobin concentration is indicative of the healthy cells in the vessel, discussed further below with respect to Tables I and II.

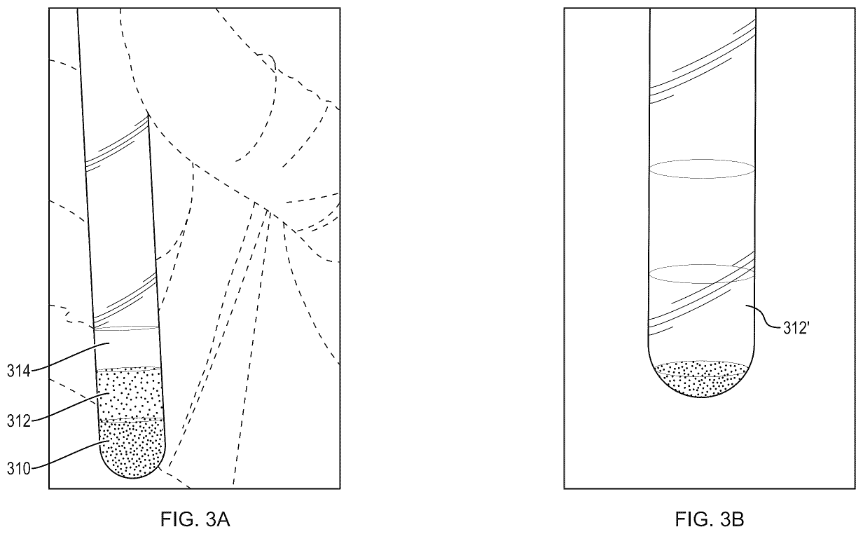

FIGS. 3A and 3B show a comparison of healthy vs degraded blood samples. Referring to FIGS. 1-3B, the disclosed device 130 monitors supercooled red blood cell samples 100 to detect occurrences of ice nucleation while minimizing sample loss, a detriment in conventional invasive testing methods for nucleation. A typical sample 100 consists of the sedimented red blood cell layer 110, the supernatant layer 112, and the layer 114 of mineral oil. When nucleation occurs, cells lyse and release hemoglobin; this causes the supernatant layer 112 of the sample to change from a clear to a red color, which can be detected using the photometer 134 based on light passage and occlusion of the light beam 136. This photometer runs continuously with the vibration sensor 158 and temperature sensor 160 in order to monitor storage conditions. FIG. 3A is an example of a stored red blood cell sample that has undergone fairly significant red blood cell lysis. The released hemoglobin from the lysed cells in the red blood cell layer 310 to migrate to the middle supernatant layer 312, denoting a distinctly pink-reddish hue from the liberated hemoglobin. Contrast this color in comparison with the non lysed sample 312′ in FIG. 3B, depicting an almost clear coloring indicative of a healthy sample with few or no lysed cells, hence no detrimental ice nucleation has occurred.

The optical parameters of the light beam 136 may be varied to correspond to a reduction or variance in illuminance received by the photometer 134 in response to hemoglobin “clouding” or obscuring light passage. In general, the illumination source 132 has a wavelength based on passage through the sample responsive to the hemoglobin presence in the sample 100. The blood cell layer 110 has cells subject to cell lysis, and the supernatant layer 112 receives hemoglobin resulting from cell lysis. The quantification of ice nucleation computed from light passage results from the supernatant layer 112 having an opacity based on cell lysis of cells in the red blood cell layer 110.

To illustrate the illuminance testing, 10 samples of water and pigmentation were made to test the photometer 134 illuminance response, listed in Table I below. The purpose of this testing was to prove that the photometer could detect color changes in samples as they progressively become more redish/pinkish. As shown in Table I, the illuminance values decreased as the concentration of pigmentation increased, which was the expected result. Less light was able to be transmitted through the sample 100 as the amount of pigment indicative of hemoglobin increased. Based on an experimental trendline, the value was calculated to be 0.996, which shows that the data is a good fit for the trendline, with no major outliers to the data. Similar results or trends can be expected for other aspects of whole-blood testing.

TABLE I

| Volume of | Average | |
|---|---|---|
| Coloring (μL) | Illuminance (lx) | Standard deviation |
| 0.00 | 24.04 | 0.05 |
| 10.00 | 22.75 | 0.38 |
| 30.00 | 21.76 | 0.17 |
| 60.00 | 20.06 | 0.10 |
| 100.00 | 18.30 | 0.03 |
| 150.00 | 16.97 | 0.00 |
| 100.00 | 18.30 | 0.03 |
| 150.00 | 16.97 | 0.00 |
| 210.00 | 14.51 | 0.06 |
| 280.00 | 13.78 | 0.08 |
| 360.00 | 12.86 | 0.02 |
| 450.00 | 11.43 | 0.06 |

Illuminance is then mapped to a hemoglobin concentration to assess the health, and thus the usability, of the blood. The concentrations of hemoglobin being tested are determined by calculating the expected range of hemoglobin that had potentially lysed from cells. There is 150 mg/ml of hemoglobin present in blood, which means that approximately 96% of a red blood cell is hemoglobin. Within a blood sample used for testing, half of which is 500 μl of red blood cells, there are 480 μg of hemoglobin. Therefore, if every red blood cell lyses, there will be 0.96 mg of hemoglobin per milliliter of blood. Using a concentration range of 0-8 mg/ml, the percent lysis was determined for each sample and is shown in Table II below.

TABLE II

| Hemoglobin Concentration | Percent Lysis |
|---|---|
| (mg/mL) | (%) |
| 0 | 0.0 |
| 0.8 | 8.3 |
| 1.6 | 16.7 |
| 2.4 | 25.0 |
| 3.2 | 33.3 |
| 4 | 41.7 |
| 4.8 | 50.0 |
| 5.6 | 58.3 |
| 6.4 | 66.7 |
| 7.2 | 75.0 |
| 8.0 | 83.3 |

Table II shows hemoglobin concentrations compared to the percentage of hemoglobin in a blood sample that has lysed. Tables I and II confirm the expected result that as the concentration increases, there is a large decrease in the illuminance values. The illuminance value for the 0 g/mL hemoglobin sample of blood was 55.012 and the respective value for the 0.0008 g/mL hemoglobin sample of blood was 30.340. As the concentration of free hemoglobin in blood increases, the illuminance value decreases. This aligns with the overall hypothesis: as the concentration of free hemoglobin in a sample increases, the illuminance values decrease.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. An opacity testing device for a liquid sample, comprising:
a vessel adapted for storing a liquid having an opacity, the vessel being transparent;
an illumination source directed at a side of the vessel;
a photometer disposed on an opposed side of the vessel from the illumination source; and
a processor connected to the photometer, the processor having nucleation logic for receiving an illuminance from the photometer, the illuminance indicative of cell lysis, and for computing a measure of healthy cells in the vessel based on the illuminance.

2. The device of claim 1, wherein the vessel is adapted to contain a sample, the sample having a plurality of layers of fluid, the illumination source and the photometer disposed for aligning the illumination source and the photometer with a sensed layer of the plurality of layers.

3. The device of claim 1, wherein the vessel is adapted to contain a sample, the opacity based on a hemoglobin presence in the sample.

4. The device of claim 1, wherein the illumination source has a wavelength based on passage through the sample responsive to the hemoglobin presence in the sample.

5. The device of claim 2, wherein the sample has a supernatant layer and a cell layer, the supernatant layer having an opacity based on cell lysis of cells in the cell layer.

6. The device of claim 2, wherein the sample includes blood, the blood having cells subject to cell lysis, and the plurality of layers includes a supernatant layer, the supernatant layer receiving hemoglobin resulting from cell lysis.

7. The device of claim 1, wherein the layers include a cell layer and a supernatant layer, the cells containing hemoglobin, wherein hemoglobin migrates to the supernatant layer upon rupture from cell lysis.

8. The device of claim 1, further comprising:
a memory, the memory coupled to the processor, and
a table of values, the table of values for receiving a value of illuminance from the photometer; and mapping the value of illuminance to a hemoglobin concentration, the hemoglobin concentration indicative of the healthy cells in the vessel.

9. The device of claim 8, further comprising:
an accelerometer; and
a temperature sensor, the memory responsive to the accelerometer and the temperature sensor for detecting physical aberrations and a temperature corresponding to the illuminance at a particular time.

10. The device of claim 5, further comprising a height control, the vessel responsive to the height control for aligning the illumination source and the photometer with the supernatant layer.

11. A method for testing health and longevity of a stored red blood sample, comprising:
storing a blood sample having an opacity in a transparent vessel;
directing an illumination source directed at a side of the transparent vessel;
receiving an illuminance at a photometer disposed on an opposed side of the vessel from the illumination source, the illuminance indicative of cell lysis; and
computing a measure of healthy cells in the vessel based on the illuminance.

12. The method of claim 11, wherein the vessel is adapted to contain a sample, the sample having a plurality of layers of liquid, further comprising aligning the illumination source and the photometer with a sensed layer of the plurality of layers.

13. The method of claim 12, wherein the illuminance results from an opacity based on a hemoglobin presence in the sample.

14. The method of claim 13, wherein the illumination source has a wavelength based on passage through the sample responsive to the hemoglobin presence in the sample.
